(19) United States
(12) Patent Application Publication
(10) Pub. No.: US 2015/0342912 A1
(43) Pub. Date: Dec. 3, 2015

(54) METHOD AND FORMULATION FOR TREATING MUCOSAL DISCONTINUITIES AND FOR REDUCING BIOFILMS

(71) Applicant: EPIEN Medical, Inc., White Bear Lake, MN (US)

(72) Inventor: Michael Basara, Hugo, MN (US)

(21) Appl. No.: 14/728,559

(22) Filed: Jun. 2, 2015

Related U.S. Application Data
(60) Provisional application No. 62/006,330, filed on Jun. 2, 2014.

(51) Int. Cl. A61K 31/185; A61K 9/00; A61K 31/60; A61K 9/06

(52) U.S. Cl. CPC A61K 31/185; A61K 9/06; A61K 9/006; A61K 9/0014; A61K 31/60

(57) ABSTRACT

A gelled formulation suitable for use in the treatment of discontinuities, comprising: phenolsulfonic acid in a concentration of 25-80% by weight; guaiacolsulfonic acid in a concentration of 25-80% by weight; free sulfuric acid in a concentration of 0 to 32% by weight; and water in a concentration of 0 to 3% by weight diluted in a gel comprising colloidal silica to a concentration of 2 to 5% by weight of the gel.

### PRIORITY CLAIM

This patent application claims priority to U.S. Provisional Patent Application Ser. No. 62/006,330, filed Jun. 2, 2014, the specification of which is incorporated herein by reference.

### FIELD

Embodiments of the invention described herein relate to a formulation for treating mucosal discontinuities and for reducing biofilms, a method for making the formulation and a method for using the formulation to treat mucosal discontinuities and to reduce biofilms.

### BACKGROUND

The term, “mucosal discontinuities” as used herein refers to discontinuities which are present or which are inflicted on mucosal tissue of living beings. Mucosal discontinuities include wounds which are internal or external bodily injuries or lesions which are caused by a physical force or by another mechanism. The physical force is one or more of a mechanical, chemical, viral, bacterial, or thermally induced physical force. The physical force disrupts the normal continuities of biologic structures of living beings.

Mucosal discontinuities include contusions, wounds in which the skin is unbroken, incisions, wounds in which the skin is broken by a cutting instrument, lacerations, and wounds in which the skin is broken by a dull or blunt instrument. Discontinuities include wounds caused by accident or by surgical procedures.

Patients who suffer major wounds and other discontinuities benefit from treatment that enhances healing and pain relief. Wound healing mechanisms comprise a series of processes whereby tissue, such as mucosal tissue, is repaired. In particular, in repair, specialized tissue is regenerated. New tissue is reorganized.

The term, “biofilm” as used herein refers to a material which naturally develops when microbes attach to a support that is made of a material including but not limited to stone, metal, plastic, glass and wood. “Biofilm” also refers to filamentous and non-filamentous bacteria that produce an extracellular polysaccharide and proteinaceous material that act as a natural glue to immobilize the cells. In nature, nonfilament-forming microorganisms stick to the biofilm surface, locating within an area of the biofilm that provides an optimal growth environment with respect to pH, eH, dissolved oxygen, and nutrients. Since nutrients tend to concentrate on solid surfaces, including porous surfaces and wet, dry surfaces, a microorganism saves energy through cell adhesion to a solid surface rather than by growing unattached.

Microbes are capable of attachment to almost any surface submerged in an aqueous environment—a phenomenon known as microbial adhesion. Colonization and proliferation of the microbes on a surface forms a biofilm.

Adhesion of microbes on a surface is involved in diseases of humans and animals, in dental plaque formation, in industrial processes, in fouling of man-made surfaces, in syntrophic and other community interactions between microorganisms, and in the activity and survival of microorganisms in natural habitats.

### SUMMARY

One embodiment includes a gelled formulation suitable for use in the treatment of oral mucosal discontinuities and other discontinuities, comprising: phenolsulfonic acid in a concentration of 25-80% by weight; guaiacolsulfonic acid in a concentration of 25-80% by weight; free sulfuric acid in a concentration of 0 to 32% by weight; and water in a concentration of 0 to 3% by weight diluted in a gel including colloidal silica to a concentration of 2 to 5% by weight.

Another embodiment includes a gelled formulation suitable for use in the treatment of skin mucosal or other discontinuities. The gelled formulation includes phenolsulfonic acid in a concentration of 25-80% by weight; ammonium phenolsulfonate in a concentration of 0 to 5% by weight; guaiacolsulfonic acid in a concentration of 25-80% by weight; free sulfuric acid in a concentration of 0 to 32% by weight; and water in a concentration of 0 to 3% by weight, the formulation diluted in a gel comprising colloidal silica to a concentration.

Another embodiment includes a gelled formulation that includes ingredients that include phenolsulfonic acid, guaiacolsulfonic acid, ammonium phenolsulfonate, potassium guaiacolsulfonate, water and free acid, formulation diluted in a gel comprising colloidal silica; and a mechanism calibrated to mix preselected amounts of two or more of the ingredients together to make a formulation suitable for use in the treatment of a specific type of discontinuity.

Another embodiment includes a method for treating a biofilm without stimulating a resistance in a living being, comprising: exposing the biofilm to a gel formulation comprising sulfonic acid derivatives of hydroxybenzenes and hydroxymethoxybenzene within a gel.

### DETAILED DESCRIPTION

Although detailed embodiments of the invention are disclosed herein, it is to be understood that the disclosed embodiments are merely exemplary of the invention that may be embodied in various and alternative forms. Specific structural and functional details disclosed herein are not to be interpreted as limiting, but merely as a basis for teaching one skilled in the art to variously employ the mucosal discontinuity treatment and biofilm removal embodiments. Throughout the drawings, like elements are given like numerals.

Referred to herein are trade names for materials including, but not limited to, polymers and optional components. The inventors herein do not intend to be limited by materials described and referenced by a certain trade name. Equivalent materials (e.g., those obtained from a different source under a different name or catalog (reference) number to those referenced by trade name may be substituted and utilized in the methods described and claimed herein. All percentages and ratios are calculated by weight unless otherwise indicated. All percentages are calculated based on the total composition unless otherwise indicated. All component or composition concentrations are in reference to the active level of that component or composition, and are exclusive of impurities, for example, residual solvents or by-products, which may be present in commercially available sources.

The term, “mucosal discontinuities” as used herein refers to discontinuities which are present or which are inflicted on mucosal tissue of living beings. Mucosal discontinuities include wounds which are internal or external bodily injuries or lesions which are caused by a physical force or by another mechanism. The physical force is one or more of a mechanical, chemical, viral, bacterial, or thermally induced physical force. The physical force disrupts the normal continuities of biologic structures of living beings. Discontinuities include mucosal discontinuities as well as contusions, wounds in which the skin is unbroken, incisions, wounds in which the skin is broken by a cutting instrument, lacerations, and wounds in which the skin is broken by a dull or blunt instrument. Discontinuities include wounds caused by accident or by surgical procedures.

In its product aspect, the present invention includes a formulation for treating discontinuities that includes phenolsulfonic acid, guaiacolsulfonic acid, for some embodiments, ammonium phenolsulfonate, free sulfuric acid and water. The formulation is derived from Beechwood creosote, and includes phenolic compounds that are isolated and purified from the Beechwood creosote. The purified phenolics obtained from the Beechwood creosote include phenol, guaiacol, salicylic acid, resorcinol and hydroquinone. The purified phenolics are treated with sulfuric acid to form sulfonic acids and sulfonate salts. The sulfonate salts are treated with one or more of zinc, potassium and sodium.

The sulfonic acids are used to treat mucosal discontinuities in the oral cavity such as aphthous stomatitis, chronic periodontitis and gingivitis. The sulfonate salts are used to treat mucosal discontinuities of the skin, such as dermatologic disorders and skin resurfacing products. When phenol is treated with sulfuric acid, the reaction forms four isomers of phenolsulfonic acid and ammonium phenolsulfonate salt. When guaiacol is treated with sulfuric acid, the reaction forms seven isomers, a mono- and a bis-form of guaiacolsulfonic acid and potassium guaiacolsulfonate.

The formulation is gelled with a pharmaceutical grade colloidal silica, AEROSIL 200 Pharma. With the gel, the formulation is usable in a concentration range of 2% to 5% by weight. The addition of the gelling agent imparts significant advantages to the Dental Professional for specific applications. In the gel form, the formulation adheres to the contact surface and does not drip or run. With this ability the formulation is applicable to an area for an extended period of time with advantages in the routine dental procedure or prophylactic treatment increasing efficiency of biofilm removal.

The gelled formulation of the present invention is a significant improvement over prior art formulations prepared with Beechwood creosote because the formulation of the present invention does not have the intense phenolic odor and taste of prior art products and is applicable to an area for an extended period of time. Furthermore, it is believed that the efficacy of the formulations of the present invention are greater than the prior art formulation because the acids and salts are separated and specifically targeted to treating the oral cavity and the skin, respectively.

One gelled formulation embodiment, for treating mucosal discontinuities of the oral cavity includes phenolsulfonic acid in a concentration of 30 percent by weight; guaiacolsulfonic acid in a concentration of 32 percent by weight; free sulfuric acid in a concentration of 24 percent by weight; water in a concentration of 11.9 percent by weight; and FD&C Red No. 40 in a concentration of 0.075 percent by weight as well as the colloidal silica gelling component.

Acceptable component ranges for a formulation for treating mucosal discontinuities of the oral cavity, such as gingivitis, are as follows:

| Component | Concentration Ranges | |
|---|---|---|
| Phenolsulfonic Acid | 25-80% | by weight |
| Guaiacolsulfonic Acid | 25-80% | by weight |
| Ammonium Phenolsulfonate | 0-32% | by weight |
| Free Sulfuric Acid | 0-32% | by weight |
| Water | 13-30% | by weight |
| Colorant | 0.075-0.020% | by weight |

### Colloidal Silica in an Amount to Gel the Other Components

Acceptable component ranges for a formulation for treating mucosal discontinuities of the skin are as follows:

| Component | Concentration Ranges | |
|---|---|---|
| Phenolsulfonic Acid | 25-80% | by weight |
| Guaiacolsulfonic Acid | 25-80% | by weight |
| Ammonium Phenolsulfonate | 0-5% | by weight |
| Free Sulfuric Acid | 0-3% | by weight |
| Water | 13-30% | by weight |
| Colorant | 0.075-0.020% | by weight |

Colloidal Silica in an amount to gel the other components.
Devices for applying the formulations of the present invention include a syringe and a cotton swab.

In its method aspect, the present invention includes a method for preparing treatments for mucosal discontinuities in the oral cavity and skin from a Beechwood creosote mixture. The method includes isolating and purifying phenolic compounds from the Beechwood creosote mixture using methods known to those skilled in the art. The method also includes treating the purified phenolics with sulfuric acid to make sulfonic acids and sulfonate salts and gelling the purified phenolics and sulfuric acid and sulfonate salts with colloidal silica.

The method further includes using the gelled sulfonic acids to treat mucosal discontinuities in the oral cavity such as aphthous stomatitis, chronic periodontitis and gingivitis. The method includes using the gelled sulfonate salts to treat dermatologic disorders and for skin resurfacing.

Since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes, which come within the meaning and range of equivalency of the claims, are intended to be embraced therein.

The gelled sulfonic acids disclosed herein are effective for removing at least about 80% of a biofilm from a surface within a period of time from substantially instantaneously to about 15 seconds. The gelled formulation includes sulfonic acid derivatives of hydroxybenzenes and hydroxymethoxybenzene in a gel comprising colloidal silica. In one embodiment, the formulation includes a quantity of four isomers of phenolsulfonic acid, seven isomers of guaiacolsulfonic acid, and for some embodiments, acid, water and a gel. The specific isomer concentrations and concentration ratios are selected by employing specific reaction parameters such as time, temperature, and concentration of reactants that produce the selected isomer concentration profile. The formulation is applied to a biofilm in a manner that covers the biofilm for about 15 seconds.

In another embodiment, the phenolsulfonic acid in a formulation for removing the biofilm is further treated with a hydroxide such as ammonium hydroxide to make gelled ammonium phenolsulfonate before application to a biofilm. The guaiacolsulfonic acid is treated with a hydroxide such as potassium or zinc hydroxide to make gelled potassium guaiacolsulfonate.

The system embodiments of the invention include formulations that include the ingredients phenolsulfonic acid, guaiacolsulfonic acid, sulfosalicylic acid, ammonium phenolsulfonate, potassium guaiacolsulfonate, water and sulfuric acid which is National Formulary (NF) in a gel that includes colloidal silica. At least one embodiment of the system also includes a mechanism calibrated to mix preselected amounts of ingredients together to make a formulation for treating a specific type of biofilm.

In its system aspect, embodiments of the invention include a gelled formulation for treating bacterial biofilms that includes phenolsulfonic acid, sulfosalicylic acid, guaiacolsulfonic acid in a gel, and, for some embodiments, ammonium phenolsulfonate, sulfuric acid and water in a gel. In one embodiment, the formulation includes phenolic compounds that are isolated and purified. The purified phenolic compounds include phenol, guaiacol, salicylic acid, resorcinol and hydroquinone. The purified phenolics are treated with sulfuric acid to form sulfonic acids and sulfonate salts. The sulfonate salts are treated with one or more of zinc, potassium and sodium to form a salt and added to a gel that includes colloidal silica. Examples of other salts include alkali metal or alkaline earth metal salts and, particularly, calcium, magnesium, sodium, lithium, zinc, potassium, and iron salts. Phenolic compounds obtained from a variety of sources are usable to make the gelled formulation described herein.

Method and system embodiments described herein are usable to remove biofilm of virtually any microbe, including but not limited to bacterial, viral, prion, fungal, cyst, of plant origin, of animal origin. For some embodiments biofilms are of two types: nonspecific and specific. Nonspecific, reversible biofilm formation is mainly a physicochemical process. A number of classes of interaction are typically involved in nonspecific biofilm formation: Van der Waals forces, dipolar, electrostatic, hydrogen bonds, hydrophobic interactions. The number and strength of these interactions vary considerably from system to system, depending on the type of the surface and microbe involved. For instance, part or an entire external surface of some microbes is hydrophobic, and the biofilm formation of some bacterial strains onto hydrophobic sulphated polystyrene should correlate with bacterial hydrophobicity. The contact angle is a relative measure for representing the degree of hydrophobicity of a surface, which in most cases shows a correlation with the surface Gibbs energy (the surface Gibbs energy decreases with an increase in hydrophobicity).

Proteins such as lectins, which are carbohydrate-binding proteins, may serve as biofilms. Lectin-like, binding sites-mediated interactions have been considered important even for bacterial adhesion to inert surfaces such as hydroxyapatite.

Formulations that include the sulfonic acids of phenol and guaiacol in a gel are used to remove biofilms on virtually any surface, including skin or other human or other living being tissue and implantable medical devices. The term “biofilm” as used herein refers to substances that contain either single or multiple microbial species and that readily adhere to such diverse surfaces as river rocks, soil pipelines, teeth, mucous membranes, and medical implants. Biofilms are biological films that can develop and persist on solid substrates in contact with moisture, on soft tissue surfaces in living organisms and at liquid air interfaces. They can develop into structures several millimetres or centimeters in thickness and can cover large surface area.

Formulations that include the phenolsulfonate salts also have use as skin resurfacing products. When phenol is treated with sulfuric acid, the reaction products include four isomers of phenolsulfonic acid. When guaiacol is treated with sulfuric acid, the reaction products include seven isomers, a mono- and a bis-form of guaiacolsulfonic acid. Other sources of phenolsulfonic acid include the four isomers. Other sources of guaical include the seven isomers, a mono- and a bis-form of guaiacolsulfonic acid.

Gel formulations prepared with Beechwood creosote are also usable for removing biofilms. One formulation embodiment, for removing biofilms is made from ingredients that include liquid phenol USP, guaiacol USP, sulfuric acid NF, purified water USP, colloidal silica for gelling, and, for some embodiments FD&C Red No. 40. The formulation includes phenolsulfonic acid in a concentration of about 30%; guaiacolsulfonic acid in a concentration of about 32%; free sulfuric acid in a concentration of about 24%; water in a concentration of about 11.9% and FD&C Red No. 400 in a concentration of about 0.075%.

The color of this formulation is purple. The total acidity is 9.1 mN/g. The density is about 1.59 g/mL. The application time is about 5 to 30 seconds. The formulation composition includes phenolsulfonic acid in a concentration of 30 percent by weight; guaiacolsulfonic acid in a concentration of 32 percent by weight; free sulfuric acid in a concentration of 24 percent by weight; water in a concentration of 11.9 percent by weight; and FD&C Red No. 40 in a concentration of 0.075 percent by weight. This formulation has a density of 1.59 g/mL, a total acidity of 9.1 mM/g, and a viscosity of 1025 cPs at 25 degrees Centigrade. The formulation does not include a tissue colorant.

Another formulation embodiment includes phenolsulfonic acid in a concentration of 25 to 80% by volume; guaiacolsulfonic acid in a concentration of 25 to 80% by volume; ammonium phenolsulfonate in a concentration of 0 to 5% by volume; free sulfuric acid in a concentration of 0 to 3% by volume; water in a concentration of 13 to 30% by volume and colorant in a concentration of 0.0075 to 0.020% by volume. Sufficient colloidal silica is added to gel the formulation.

The gelled formulation embodiments are applied to surfaces having a biofilm with an applicator, for some embodiments. The application contact time for treatment ranges from less than 1 to about 15 seconds for removal of at least about 80% of the biofilm from the surface.

Acceptable component ranges for formulation embodiments for removing biofilms from a surface, are as follows:

| Component | Concentration Ranges | |
|---|---|---|
| Phenolsulfonic Acid | 25-80% | by weight |
| Guaiacolsulfonic Acid | 25-80% | by weight |
| Sulfosalicylic Acid | 0-30% | by weight |
| Citric Acid | 0-30% | by weight |
| Ammonium Phenolsulfonate | 0-32% | by weight |
| Free Sulfuric Acid | 0-32% | by weight |
| Water | 13-30% | by weight |

Colloidal Silica Sufficient to Gel

The total acidity is 7.20 to 9.20 mM/g. The density is 1.46 to 1.59 g/mL. The application time is less than 1 second to not more than about 15 seconds. The applicator includes in some embodiments, a vial, a pre-filled syringe, a swab, a patch or combinations of these applicators. The formulation optionally includes a colorant such as green or blue.

Devices for applying the formulations described herein are shown in FIG. 7 and include in one embodiment, a syringe and a cotton swab. In one embodiment, the syringe is prefilled. In one embodiment, formulation embodiments are enclosed within a swab device. In another embodiment, the formulation is enclosed in a container with a delivery mechanism that delivers the formulation drop-by-drop. In another embodiment, the formulation is metered. In one other embodiment, the formulation is enclosed in a container that includes a mechanism for heating or cooling the formulation. The mechanism includes chemicals, separated in compartments by a breakable seal, adjacent to the formulation embodiments described herein, that create an exothermic reaction when combined. In one embodiment, the chemicals are separated from the formulation by a wall. The chemicals are combined when a user breaks the breakable seal. The heating is transferred to the formulation through the wall. The packaged formulation embodiments are capable of being transported for sale to pharmacies and drug stores.

In another embodiment, formulation embodiments described herein are usable for sterilizing devices and components used in applications that include but are not limited to medical and dental applications. For some embodiments, formulations are passed through tubing to sterilize the tubing. For other embodiments, formulations are used to sterilize dental and medical equipment, such as catheters.

For some embodiments, formulation embodiments are usable to remove biofilm from seeds. Formulation embodiments are also usable to remove biofilms that form along inner walls of conduits in industrial facilities and in household plumbing systems. Formulation embodiments are also useful to treat biofilms in cooling water systems used in power-generating plants, refineries, chemical plants, and air conditioning systems. Cooling water systems are often contaminated with airborne organisms entrained by air/water contact in cooling towers as well as waterborne organisms from the system's makeup water supply. Formulation embodiments are also usable to treat biofilms that form in water supply storage and conveying equipment.

Formulation and method embodiments are also usable to remove biofilms from locations that have implications in human and animal health Biofilms can present a serious threat to health as foci of chronic infections. For example, biofilm composed of Pseudomonas aeruginosa, the bacterium responsible for biofilm formed in the lungs of cystic fibrosis patients, is believed to be behind the fatal lung infections in patent suffering this disease. Biofilms have been implicated in periodontal disease, tooth decoy, prostate infections, kidney stones, tuberculosis. Legionnaire's disease and some infections of the middle ear.

Gelled formulations and methods described herein are also usable to prevent infections resulting from medical intervention. For example, formulations and methods described herein can remove biofilms that form on medical devices including catheters, medical implants, dental equipment and contact lenses.

Gelled formulations and method embodiments described herein are usable to prevent biofilm formation in bioimplants such as bone prosthesis, heart valves, pacemakers, stents, orthopaedic devices, ear implant devices, electrodes, dialysis devices and the like.

The biofilm treatment embodiments described herein have a benefit over antibiotic use in that organisms forming biofilm, ranging from prions to viruses, to bacteria to fungi, to cysts, and any other biofilm formers, do not develop a resistance to the biofilm formulations.

Gelled formulations described herein are believed to work by mechanisms of therapeutic action that include solvent/keratolytic; hygroscopic/dehydrating/and denaturant/keratocoagulant. The solvent/keratolytic mechanixm penetrates and dissolves necrotic tissue. The hygroscopic/dehydrating mechanism reduces tissue edema. Solvation of the formulation is exothermic and releases an acidification mechanism. The denaturant/keratocoagulant mechanism produces acidification necrosis and oxidation and surrogate eschar/clot which produces a protective natural bandage.

Gelled formulation embodiments described herein impart virtually instantaneous pain relief; accelerated ulcer healing; an infectious organism kill; and effectiveness in a one-time treatment. The gelledformulation embodiment acts by a self-limited action that is not harmful to healthy mucosa. The gelled formulation forms a barrier membrane and neutralize acid.

Since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes, which come within the meaning and range of equivalency of the claims, are intended to be embraced therein.

What is claimed is:

1. A gelled formulation suitable for use in the treatment of oral mucosal discontinuities, comprising: phenolsulfonic acid in a concentration of 25-80% by weight; guaiacolsulfonic acid in a concentration of 25-80% by weight; free sulfuric acid in a concentration of 0 to 32% by weight; and water in a concentration of 0 to 3% by weight diluted in a gel comprising colloidal silica to a concentration of 2 to 5% by weight of the gel.

2. A gelled formulation suitable for use in the treatment of skin discontinuities, comprising: phenolsulfonic acid in a concentration of 25-80% by weight; ammonium phenolsulfonate in a concentration of 0 to 5% by weight; guaiacolsulfonic acid in a concentration of 25-80% by weight; free sulfuric acid in a concentration of 0 to 32% by weight; and water in a concentration of 0 to 3% by weight, the formulation diluted in a gel comprising colloidal silica to a concentration of 2 to 5% by weight of the gel.

3. A kit suitable for use in the treatment of discontinuities comprising the gelled formulation of claim 1 or 2, a container for containing the gelled formulation and a device for delivering the gelled formulation to a mucosal discontinuity.

4. The gelled formulation of claim 2, further comprising one or more of salicylic acid and sulfosalicylic acid.

5. A formulation for removing a biofilm from a surface, comprising a formulation comprising phenolsulfonic acid guaiacolsulfonic acid, sulfosalicylic acid, citric acid, and ammonium phenolsulfonate a gel comprising colloidal silica, wherein the phenolsulfonic acid guaiacolsulfonic acid, sulfosalicylic acid, citric acid, and ammonium phenolsulfonate are within the gel and an applicator for applying the gel to a biofilm for at least about 15 seconds.
